(19) United States
(12) Patent Application Publication
(10) Pub. No.: US 2009/0226483 A1
(43) Pub. Date: Sep. 10, 2009

(54) SUPER BROWN CREAM

(76) Inventor: Zdenko Glavas, Nashville, TN (US)

Correspondence Address: James Ray & Associates, 2640 Pitcairn Road, Monroeville, PA, 15146, US

(21) Appl. No.: 12/416,426

(22) Filed: Apr. 1, 2009

Related U.S. Application Data
(63) Continuation-in-part of application No. 12/317,769, filed on Jan. 27, 2009.
(60) Provisional application No. 61/068,664, filed on Mar. 10, 2008.

(51) Int. Cl. A61K 36/00; A61P 43/00

(52) U.S. Cl. 424/195.18

(57) ABSTRACT

A topical cream for use in healing hemorrhoids, tumid veins, damaged capillaries, rectal bleeding and rectal discomfort. The cream comprises propolis that has been extracted from material formed by honeybees, such propolis is present in the cream in a predetermined amount. Olive oil is blended with such propolis in a predetermined ratio to form such topical cream.

### CROSS REFERENCE TO RELATED APPLICATIONS

This application is a Continuation-in-Part and claims benefit from pending U.S. application Ser. No. 12/317,769 filed Jan. 27, 2009.

### FIELD OF THE INVENTION

The present invention relates, in general, to a topical cream, and, more particularly, the present invention relates to a topical cream that can be used for a variety of applications and that such cream includes propolis and olive oil.

### BACKGROUND OF THE INVENTION

Many people are bothered by hemorrhoids, tumid veins, damaged capillaries, rectal bleeding and other rectal discomforts. There are hemorrhoid creams and suppositories presently on the market; however, there are very few products that are available for rectal bleeding and rectal discomfort.

Thus, it would be advantageous if there were a simple and inexpensive topical cream that could be used to heal rectal problems including rectal bleeding, rectal discomfort, hemorrhoids, tumid veins and damaged capillaries.

### SUMMARY OF THE INVENTION

In a first aspect the present invention provides a topical cream for use in healing hemorrhoids, tumid veins, damaged capillaries, rectal bleeding and rectal discomfort. The cream comprises propolis that has been extracted from material formed by honeybees, such propolis is present in the cream in a predetermined amount and olive oil is blended with such propolis in a predetermined ratio.

In a second aspect the present invention provides a method for preparing a topical brown cream for use in healing hemorrhoids, tumid veins, damaged capillaries, rectal bleeding and rectal discomfort. The method comprises the steps of obtaining some propolis pitch and then grating a predetermined amount of the propolis pitch obtained in the previous step. There is a step of diluting ethyl alcohol to make a 60 per cent solution and then mixing such predetermined amount of propolis obtained in a previous step with a predetermined amount of the prepared ethyl alcohol. There is a step of storing the prepared mixture prepared for about fourteen days and a step of stirring the mixture twice a day fort each day that such mixture is stored and a step of separating said propolis from sediment in the mixture. Then there is a step of placing such propolis separated from the mixture in a dark bottle and mixing a first predetermined amount of the propolis disposed in such dark bottle with a predetermined amount of olive oil. There is a step of keeping the mixture prepared in the previous step for about six days until a bottom portion is a cream with some olive oil and alcohol above the cream and a step of removing the cream from the mixture and finally a step of applying the cream containing propolis and olive oil removed in the previous step to a rectum for healing such hemorrhoids, tumid veins, damaged capillaries, rectal bleeding and rectal discomfort.

### OBJECTS OF THE INVENTION

It is, therefore, one of the primary objects of the present invention to provide a topical cream for the treatment of hemorrhoids, tumid veins, damaged capillaries, rectal bleeding and rectal discomfort.

Another object of the present invention is to provide a topical cream that includes propolis.

Still another object of the present invention is to provide a topical cream that further includes olive oil.

Yet another object of the present invention is to provide a method for the preparation of such topical cream.

In addition to the various objects and advantages of the invention which have been described in some specific detail above it should be noted that various other objects and advantages of the present invention will become more readily apparent to those persons who are skilled in the relevant art from the following more detailed description, particularly when such description is taken in conjunction with the appended claims.

### BRIEF DESCRIPTION OF THE PRESENTLY PREFERRED AND VARIOUS ALTERNATIVE EMBODIMENTS OF THE INVENTION

In a first aspect the present invention provides a topical cream for use in healing hemorrhoids, tumid veins, damaged capillaries, rectal bleeding and rectal discomfort. The cream comprises propolis that has been extracted from material formed by honeybees, such propolis is present in the cream in a predetermined amount and olive oil is blended with such propolis in a predetermined ratio.

The ratio of such propolis and such olive oil in preparing the cream blend is about one to one to form such brown cream. It is presently preferred that the mixture comprises four spoonfuls of propolis and about four spoonfuls of olive oil.

In a second aspect the present invention provides a method for preparing a topical brown cream for use in healing hemorrhoids, tumid veins, damaged capillaries, rectal bleeding and rectal discomfort. The method comprises the steps of obtaining some propolis pitch and then grating a predetermined amount of the propolis pitch obtained in the previous step. There is a step of diluting ethyl alcohol to make a 60 per cent solution and then mixing such predetermined amount of propolis obtained in a previous step with a predetermined amount of the prepared ethyl alcohol. There is a step of storing the prepared mixture prepared for about fourteen days and a step of stirring the mixture twice a day fort each day that such mixture is stored and a step of separating said propolis from sediment in the mixture. Then there is a step of placing such propolis separated from the mixture in a dark bottle and mixing a first predetermined amount of the propolis disposed in such dark bottle with a predetermined amount of olive oil. There is a step of keeping the mixture prepared in the previous step for about six days until a bottom portion is a cream with some olive oil and alcohol above the cream and a step of removing the cream from the mixture and finally a step of applying the cream containing propolis and olive oil to a rectum for healing such hemorrhoids, tumid veins, damaged capillaries, rectal bleeding and rectal discomfort.

The method further includes a step of obtaining two ounces of grated propolis from such propolis pitch and another step of mixing four ounces of such propolis that was disposed in the dark bottle with about four ounces of olive oil obtained in a previous step. The method also includes a step of adding about 8 ounces of such 60 per cent alcohol to such two ounces of grated propolis.

Propolis is a substance made by the honeybee that provides protection against harmful bacteria, viruses and fungi. It is a plant resin collected by bees for use in and around the hive. In plants it is usually a sticky coating around buds that serves to protect them from the elements of weather plus from attack by bacteria, fungi, molds and viruses. These are properties that are useful to the bees and are enhanced by the sticky properties of the propolis beeglue. Like pollen, propolis beeglue is a bee product that cannot be clearly defined and varies from sample to sample. This is a natural outcome of the collection process. Propolis collecting bees will use resins from a large variety of tree and other plant species. Nevertheless, different bee propolis extracts samples do share considerable similarity in their physical and overall general nature.

Hundreds of chemical compounds have been identified from bee propolis extract. The main chemical classes present in propolis are flavonoids, phenolics, and various aromatic compounds. Bee propolis also contains some volatile oils, terpenes and beeswax but these compounds are not believed to contribute as significantly to the chemical properties and effects of propolis. Flavanoids are well-known plant compounds that have antioxidant, anti-bacterial, anti-fungal, anti-viral and anti-inflammatory properties. Other properties of propolis beeglue include acting as a local anesthetic, reducing spasms, healing gastric ulcers and strengthening capillaries.

Thus, as can be seen propolis is a product with unlimited capabilities. However, great care must be used in preparing the product which contains propolis. Also there must be care in persons that use it such as people that allergic to bee stings. Although it doesn't mean that a person ho is allergic to bee stings cannot take propolis, but any bee product can cause an allergic reaction to a susceptible individual.

While a presently preferred embodiment and alternate embodiments of the present invention have been described in detail above, it should be understood that various other adaptations and/or modifications of the invention can be made by those persons who are particularly skilled in the art without departing from either the spirit of the invention or the scope of the appended claims.

I claim:

1. A topical cream for use in healing hemorrhoids, tumid veins, damaged capillaries, rectal bleeding and rectal discomfort, said cream comprising:
(a) propolis that has been extracted from material formed by honeybees, said propolis is present in said cream in a predetermined amount; and
(b) olive oil blended with said propolis in a predetermined ratio.

2. The topical cream, according to claim 1, wherein said ratio of said propolis and said olive oil in preparing said cream blend is about one to one.

3. The topical cream, according to claim 1, wherein said topical cream is a brown cream.

4. The topical cream, according to claim 1, wherein between about 3¾ spoonfuls and about 4¼ spoonfuls of propolis are blended with about 3¾ and about 4¼ said olive oil in said topical cream.

5. A method for preparing a topical brown cream for use in healing hemorrhoids, tumid veins, damaged capillaries, rectal bleeding and rectal discomfort, said method comprising the steps:
(a) obtaining some propolis pitch;
(b) grating a predetermined amount of propolis pitch obtained in step (a);
(c) diluting ethyl alcohol to make a 60 per cent solution;
(d) mixing said predetermined amount of propolis obtained in step (b) with a predetermined amount of ethyl alcohol prepared in step (c);
(e) storing said mixture prepared in step (d) for about fourteen days;
(f) stirring said mixture twice a day each day such mixture is stored;
(g) separating said propolis from sediment in said mixture
(h) placing said propolis separated from said mixture in a dark bottle;
(i) mixing a first predetermined amount of said propolis disposed in said dark bottle with a second predetermined amount of olive oil;
(j) keeping said mixture prepared in step (i) for about six days until a bottom portion is a cream with some olive oil and alcohol above said cream;
(k) removing said cream from said mixture;
(1) applying said cream removed in step (k) to a rectum for healing said hemorrhoids, tumid veins, damaged capillaries, rectal bleeding and rectal discomfort.

6. The method, according to claim 5, wherein said method further includes a step of obtaining about two ounces of grated propolis from said propolis pitch.

7. The method, according to claim 5, wherein said method includes the step of mixing about four ounces of propolis that was disposed in said dark bottle with about four ounces of olive oil in step (i).

8. The method, according to claim 5, wherein said method further includes a step of adding about 8 ounces of said 60 per cent alcohol to such 2 ounces of grated propolis in step (d).
